(19) United States
(12) Patent Application Publication
(10) Pub. No.: US 2022/0118114 A1
(43) Pub. Date: Apr. 21, 2022

(54) Manganese Ferrite Nanoparticles for Use as MRI Contrast Agents and Magnetohypothermia Agents

(71) Applicant: Northeastern University, Boston, MA (US)

(72) Inventors: Parisa ANDALIB, Malden, MA (US); Vincent G. HARRIS, Sharon, MA (US)

(21) Appl. No.: 17/421,607

(22) PCT Filed: Jan. 9, 2020
(86) PCT No.: PCT/US2020/012997

§ 371 Date: Jul. 8, 2021

Related U.S. Application Data
(60) Provisional application No. 62/790,450, filed on Jan. 9, 2019.

(51) Int. Cl. A61K 49/18; A61K 41/00; A61K 9/16; A61K 47/69; A61K 47/54

(52) U.S. Cl. CPC A61K 49/1827; A61K 41/0052; A61K 47/545; A61K 47/6929; A61K 9/1688

(57) ABSTRACT

The present disclosure is directed to methods of Quantum Spin Engineering of spinel superparamagnetic ferrite nanoparticles (SMFNs) for MRI contrast agents and for magnetohyperthermia agents. Using the methods herein, the magnetic properties of the SMFNs can be controlled by changing the amount of 3d-transition element cations having unpaired electrons in the 3d orbital that occupy the octahedral sites of the spinel crystal form, to form mixed spinels, while anions in the spinels can be utilized to magnetically couple the cations utilizing intra-crystalline angles determined by ion sizes and crystal structure, and further tuning of other critical parameters is provided. The mixed spinels disclosed herein provide enhanced MRI contrast agents and improved magnetohyperthermia agents with lower toxicity and safety concerns, while the production methods disclosed herein have lower cost.

### CROSS-REFERENCE TO RELATED APPLICATIONS

This application claims priority to U.S. Provisional Application No. 62/790,450, filed 9 Jan. 2019, the entirety of which is incorporated herein by reference.

### BACKGROUND

Chelated organic gadolinium complexes are currently used as gadolinium-based MRI contrast agents (CAs) in medical MRI. Before administration to patients, gadolinium (III) is chelated using various types of ligands. However, there are growing concerns over the toxicity of gadolinium (Gd) contrast agents. After administration to patients, gadolinium can separate over time from the chelating agents and can cause acute or chronic symptoms in patients. Therefore, there is a growing need for advanced superparamagnetic MRI contrast agents without the safety concerns of gadolinium.

The toxicity of Gd-based CAs leads to complications resulting in not only serious illness but possibly the death of patients and many millions of dollars in unnecessary patient care. Finding safe alternative to Gd-based CA for patients for ultrahigh resolution of early stage tumors would provide an outstanding benefit to society.

Medical diagnosis of many diseases is only possible with the use of magnetic resonance imaging (MRI). According to the Organization for Economic Cooperation and Development, the utilization rate for MRI in 2016 was reported to be 12% of the population of the United States (i.e. ˜38 million MRI scans) among which approximately 70% of those (i.e. ˜27 million) were performed using magnetic contrast agents. According to ResearchandMarkets.com, the market size for MRI contrast agents (CA) has been estimated to be $4.5 billion dollars in 2017 growing at a compound annual growth rate of 3.9%. The global MRI contrast agent market size is expected to reach an approximate value of USD 5.8 billion by 2026, according to a report by Grand View Research, Inc. It is projected to expand at a CAGR of 5.3% during this forecast period.

After Gd separates from chelating agents, it can accumulate in tissues. Although Gd based CA deposition in the brain has gained substantial attention, animal studies have revealed up to 100 times more Gd residuals are retained in skin and bones [12]. Health risks caused by Gd separation from the chelated Gd contrast agents include fatal diseases such as nephrogenic systemic fibrosis (NSF). There are two classes of ligands (i.e. linear and macrocyclic) being used for chelation of the Gd. The linear agents have the higher risk of leaving Gd ion residuals behind due to their lower stability. While the macrocyclic-type ligands exhibit lower levels of Gd deposition, they remain a subject of medical concern.

The magnitude of this issue is more significant for groups of renally impaired patients in need of multiple follow-up MRIs, such as cancer patients who require repeated imaging to monitor the tumor treatment progress. Inefficient renal excretion of these Gd particles and the accumulation of the Gd depositions, respectively, are some reasons for the elevated scale of the deleterious side effects resulting from the Gd release. This is in spite of the indispensable need for these agents in the detection of tumors where imaging sensitivity is an ongoing challenge. Therefore, oncology is one of the communities, among many, that is highly dependent on MRI diagnostics of immunosuppressed patients.

Following the concerns of Gd deposition, in July 2017, the European Medical Agency (EMA) issued the suspension of the marketing and sale of linear Gd-based CAs, except for two which remain in the market with restricted dosages to be used with extreme caution. The EMA stated that “The intravenous linear agents gadoxetic acid and gadobenic acid can continue to be used for liver scans because they are taken up in the liver and meet an important diagnostic need” [www.ema.europa.eu/medicines/human/referrals/gadolinium-containing-contrast-agents].

In December 2017, the U.S. Food and Drug Administration also issued safety measures for cautious administration of Gd contrast agents by healthcare professionals, only for cases wherein no alternative technique is available for the diagnosis of the disease and always to be used at the lowest possible dose resulting in sufficient contrast enhancement. The FDA also asked for a new warning statement to be included in the drug safety warnings and asked for educating the patients about the risk-benefit balance. This is while the contrast agent industry was asked to conduct further clinical studies on the safety of the agents to resolve the Gd health concerns. The recently updated protocols, i.e. the reduction or elimination of some of the MRI contrast agents, resulted in limitations for the oncology community, among others, compromising the diagnosis and treatment of diseases. The shortcoming of the remaining CAs in the market for the diagnosis of liver cancer has given rise to a market gap in the MRI industry during the past year.

The existing nontoxic Gd-free contrast agent markets are presently dominated by superparamagnetic iron oxide nanoparticles (SPIONs). These products include Ferumoxides (Feridex® IV, Berlex Laboratories), Ferucarbotran (Resovist®, Bayer Healthcare), Ferumoxtran-10 (AMI-227 or Code-7227, Combidex®, AMAG Pharma, Sinerem®, Guerbet), NC100150 (Clariscan®, Nycomed,) and (VSOP 0184®, Ferropharm). SPIONs do not suffer from deleterious side effects but cannot compete in performance with Gd contrast agents.

### SUMMARY

The present technology relates to compositions, systems, and methods for providing advanced contrast agents for MRI and for targeted magnetohyperthermia cancer remediation. Compared to the toxicity of Gd-based CAs (GBCAs), iron oxide, being a highly biocompatible material, is a safe option among magnetic particles for use as CAs. Residual iron oxide nanoparticles can safely enter metabolic processes, as well as the excess amount being naturally managed and excreted by the body. Iron oxide nanoparticles have been approved by the FDA for various medical applications such as MRI, drug delivery, and magnetohyperthermia cancer treatment. However, the available iron oxide nanoparticles are not as effective contrast agents when compared to the Gd complexes used for MRI contrast agents.

A very high spin-spin relaxation value of 218 mM⁻¹S⁻¹ originating from the strong magnetization (Ms=101 emu g⁻¹) of iron oxide nanoparticles, causing rapid dephasing of the nuclei, makes them more suitable as a T₂ contrast agent. Contrast agents take advantage of strong contrast enhancement that is directly dependent on Ms, with spin-spin relaxation (r2) being approximately proportional to the square of Ms.

Methods are described herein to provide advanced MRI contrast agents, for example, by cation, Quantum Spin Engineering (QSE), which enables tuning of the properties of nanoparticles for various advanced MRI and treatment applications through highly detailed tuning of multiple aspects of synthetic conditions. The transition metal manganese (Mn) possesses the second highest paramagnetic moment of any element, after gadolinium. This derives from the Mn⁺² ion having 5 unpaired electrons in its 3d shell. Herein, using manganese in a (super)paramagnetic contrast agent can be accomplished in a stable crystal structure. Using cation spin engineering and a spinel crystal structure, manganese-ferrite nanoparticle (NP) contrast agents (CAs) can serve as replacements for Gd-complexes. Mn²⁺ has a very high magnetic moment (the five unpaired d-orbital electrons are equal to 5 Bohr magnetons), while also being an essential element in body metabolism. Substitution of Fe²⁺ with Mn²⁺ in spinel crystal can enhance the r2 value from 218 mM⁻¹S⁻¹ to 358 mM⁻¹S⁻¹ (for a particle size of 12 nm)[11].

In addition to utilization for MRI contrast agents, the magnetic nanoparticles of the present technology can used for magnetohyperthermia therapy of cancer. The hyperthermia treatment can kill cancerous cells by elevating their temperatures to the therapeutic temperature range of 42-45° C. for several minutes to induce necrosis in the cancer cells. Using cation or quantum spin engineering, the same magnetic nanoparticles can be utilized for magnetohyperthermia and for MRI contrast agents. Design of magnetic nanoparticles that can serve as both MRI contrast agents and MHT agents can provide the additional benefits to patients of being minimally invasive and MHT efficient. MHT treatment is preferably performed after targeting and delivery of the nanoparticles to an affected organ.

Superparamagnetic nanoparticles (SPM NPs) of the present technology are preferably functionalized for tumor-targeting. Attaching cancer cell binding agents to the nanoparticles enables the targeting of both specific chemical profile and physical collocation. This approach enables treating small tumors that might otherwise be missed while at the same time, tumor-targeted MRI imaging benefits from high anatomic resolution and contrast. Compositions of the present technology can be suitable for targeting, imaging, and therapy of cancer cells in order to address the urgent market need for CA for MRI imaging of the affected organs of cancer patients.

Various techniques can be used for targeting SPM NPs to tumors. Passive targeting relies on physiological abnormalities of the tumor tissue in contrast to healthy cells, e.g., using the phagocytic activity of the healthy liver Kupffer cells, which the hepatocellular carcinomas (HCC) lack. However, passive targeting does not provide high contrast-to-noise ratio imaging of the tumor compared to active targeting due to interference from the inflammation in the tissue. This is in spite of the simplicity of the surface modifications (e.g., polymer coating on the SPM NPs) used in passive targeting. In active targeting techniques, targeting ligands (e.g., RGD peptides, folate molecules, mAbs, and aptamers) can be covalently attached to polymer-coated SPM NPs, can actively track specific tumor markers (e.g., liver cancer markers such as alpha-fetoprotein (AFP) and carcinoembryonic antigen (CEA)), and can bind to a tumor, followed by endocytosis by the cancer cells.

The conjugation of SPM NPs to tumor-specific molecules can be used for functionalization (targeting). This approach can include the application of peptides with high affinity for the integrins in tumor endothelia (e.g., αᵥβ₃ and αᵥβ₅,); the peptides can be RGD (arginylglycylaspartic acid) peptides. The SPM NPs can be dispersed in an organic surfactant (e.g., oleic acid) in a preparation for conjugation to biomolecules, as well as amphiphilic polymers (e.g., poly(maleic anhydride-alt-1-octadecene (PMAO)) encapsulating the SPM NPs for the formation of oil-in-water micelles to address the stability requirements. This intermediate layer can prepare the surface for linking to RGD peptides [Yu et. al. 2006, Arriortua et. al. 2016; See FIG. 1, which shows a schematic illustration of a functionalization process for SPM NPs for liver tumor targeting].

The liver metastases receiving the RGD functionalized cation spin engineered Mn(Me)-ferrite via the hepatic arteries of animals tested can be scanned using a Bruker BioSpec 70/20 MRI scanner equipped with superconducting magnet, capable of producing a magnetic field of up to 7 tesla (T).

The present technology can provide Quantum Spin Engineered ferrite nanoparticles (NPs), for example, Mn(Me)-ferrite (Me=3d-transition element), Zn—Mn—Fe-oxide NPs, Zn—Fe-oxide NPs, or Mn—Fe-oxide NPs (MFONPs), as well as compositions containing them, which are non-toxic, or exhibit low levels of toxicity, allowing for higher dosages, higher imaging efficacy, and magnetohyperthermia treatment techniques. These NPs are cation spin-engineered superparamagnetic nanoparticles for MRI contrast agents and/or targeted magnetohyperthermia cancer remediation. The NPs can include, for example, Cr⁺², Zn⁺², specific crystallinity, microstructure, morphology, phase, size, monodispersity, tumor/disease targeting complexes, and functionalized coatings.

Methods for production of the NPs can include, for example, cation spin engineering or Quantum Spin Engineering (QSE), which enables tuning of the properties of the nanoparticles for various MRI applications. QSE or cation spin engineering can, for example, tailor nanoparticle size, magnetization, spin-spin relaxation, magnetic anisotropy energy, and Neel relaxtion. The methods for production can involve substitution of Zn²⁺ for the Mn²⁺ wherein the Zn cations reside solely on the tetrahedral sublattice and displace Mn²⁺ to octahedral (high spin) sites.

Methods of conjugating the nanoparticles (NPs) with tumor-specific molecules are discussed herein. For example, when conjugated with appropriate integrin receptor ligands, Mn—Fe-oxide NPs (MFONPs) can be targeted to specific cancers, enabling a method for enhanced imaging of specific cancerous regions where MFONPs are collocated.

Methods of treatment are discussed herein. For example, being magnetic, once the MFONPs are located to cancerous regions of the body, an alternating current (AC) magnetic excitation field can stimulate the particles giving rise to localized heating and bringing about cancer cell necrosis preferentially to healthy cell necrosis. This process is referred to as magnetohyperthermia therapy (MHT).

Simultaneous imaging and treatment methods can be done utilizing the NPs herein. Such methods include, for example, methods in which magnetic MFONPs can be used simultaneously as contrast agents to image the MHT region. Simultaneously the contrast agents can be used as therapy agents.

The NPs described herein can provide non-toxic methods of MRI imaging enhancement, with imaging capabilities comparable to methods using toxic Gd-complexes, can provide methods for early detection of cancer, and can provide methods to destroy cancer cells while leaving healthy cells unaffected.

Importantly, the NPs discussed herein have low toxicity compared to previous contrast agents (CAs) that are based on Gd-complexes. The NPs can be made having the appropriate diameter, size distribution, and shape necessary for images and MHT applications without detrimental clustering of the NPs. As MRI magnetic fields increase in modern MRI instrumentation, the NPs herein can offer superior contrast imaging in comparison to existing Gd-complexes in the marketplace.

As used herein, a “3d-transition element” is an element whose atom has an incomplete d sub-shell, or which can give rise to cations with an incomplete d sub-shell, for example, a 3d-transition metal.

As used herein, the term “nanostructure” refers to a structure having at least one dimension on the nanoscale, that is, at least one dimension from about 0.1 nm to 1000 nm, preferably from about 0.1 nm to about 100 nm, or from about 1 nm to about 100 nm. Nanostructures can include, but are not limited to, nanowires, nanotubes, nanoparticles (e.g., hexagonal, triangular, or spherical shaped nanoparticles, also called nanospheres), nanosheets, nanorods, and combinations thereof. Aggregated nanostructures may comprise a plurality of multi-shaped nanostructures. A nanostructure may comprise a thickness on the nanoscale with a length on the micro or millimeter scale.

As used herein, “superparamagnetism” refers to nanoparticles in a paramagnetic state while below the Curie temperature of the nanoparticles. In the superparamagnetic state an external magnetic field is able to magnetize the nanoparticles, similarly to a paramagnet. The magnetic susceptibility of superparamagnetic nanoparticles is higher than paramagnets. In sufficiently small nanoparticles, magnetization can randomly flip direction under the influence of temperature, and the Neel relaxation time is the typical time between two flips. When the time used to measure the magnetization of the nanoparticles, without the presence of an external magnetic field, is much longer than the Neel relaxation time, the magnetization of superparamagnetic nanoparticles appears to be zero on average. As used herein, Quantum Spin Engineering can refer to cation spin engineering wherein cation high spin states are magnetically coupled in crystal structures to optimize MRI contrast agents and magnetohyperthermia effectiveness of nanoparticles comprising the crystal structures by designed syntheses.

The present technology can be further summarized by the following features:

1. Superparamagnetic manganese ferrite nanoparticles comprising a spinel compound having a crystal structure represented by

[Mn_{1-δ}^{+2,+3}+Fe_{δ}^{+2,+3}]ₓ^{Tet}{Fe_{1-δ}^{+2,+3}+Mn_{δ}^{+2,+3}}_{y}^{Oct}O₄

wherein δ is a cation inversion parameter representing a population of Mn ions on octahedral sites; wherein x is 0.8 to 1.2, y is 1.8 to 2.2, and x+y=3; wherein Fe^{+2, +3} comprises Fe⁺² or Fe⁺³, and wherein Tet designates a spinel tetrahedral site and Oct designates a spinel octahedral site.
2. Superparamagnetic zinc ferrite nanoparticles comprising a spinel compound having a crystal structure represented by

[Zn_{1-ϕ}⁺²+Fe_{ϕ}^{+2,+3}]ₓ^{Tet}{Fe_{1-ϕ}^{+2,+3}+Zn_{ϕ}⁺²}_{y}^{Oct}O₄

wherein ϕ is a cation inversion parameter representing a population of Zn ions on octahedral sites; wherein x is 0.8 to 1.2, y is 1.8 to 2.2, and x+y=3; wherein Fe^{+2, +3} comprises Fe⁺² or Fe⁺³, and wherein Tet designates a spinel tetrahedral site and Oct designates a spinel octahedral site.
3. Superparamagnetic manganese zinc ferrite nanoparticles comprising a spinel compound having a crystal structure represented by

[Zn⁺²_{1-ϕ}+Mn_{1-δ-ϕ}^{+2,+3}+Fe_{δ-ϕ}^{+2,+3}]ₓ^{Tet}{Fe_{1-δ-ϕ}^{+2,+3}+Mn_{δ-ϕ}^{+2,+3}Zn⁺²_{ϕ}}_{y}^{Oct}O₄

wherein δ is a cation inversion parameter representing a population of Mn ions on octahedral sites; wherein ϕ is a cation inversion parameter representing a population of Zn ions on octahedral sites; wherein x is 0 to 1.25, y is 0.75 to 3, and x+y=3; wherein Fe^{+2, +3} comprises Fe⁺² or Fe⁺³, and wherein Tet designates a spinel tetrahedral site and Oct designates a spinel octahedral site.
4. Superparamagnetic mixed ferrite nanoparticles comprising a spinel compound having a crystal structure represented by

[AM1⁺²_{1-ϕ}+DM2_{1-δ-ϕ}⁺²+(1−D)M2_{1-δ-ϕ}⁺³+EFe_{δ-ϕ}⁺²+(1−E)Fe_{δ-ϕ}⁺³]ₓ^{Tet}{GFe_{1-δ-ϕ}⁺²+(1−G)Fe_{1-δ-ϕ}⁺³+JM2_{δ-ϕ}⁺²+(1−J)M2_{δ-ϕ}⁺³+(1−A)M1⁺²_{ϕ}}_{y}^{Oct}O₄

wherein M1 is a divalent cation, such as a cation of Zn, Cu, Mg, or Cr;
wherein M2 is a mixed valence 3d transition metal cation, such as a cation of a transition metal such as Mn, Fe, Ni, or Co;
wherein A=0 to 1, (D+J)=0 to 1, (E+G)=0 to 1, and x+y=3;
wherein δ is a cation inversion parameter representing a population of Mn ions on octahedral sites;
wherein ϕ is a cation inversion parameter representing a population of Zn ions on octahedral sites;
wherein Fe^{+2, +3} comprises Fe⁺² or Fe⁺³, and wherein Tet designates a spinel tetrahedral site and Oct designates a spinel octahedral site.
5. The superparamagnetic mixed ferrite nanoparticles of feature 4, wherein M1 is selected from the group consisting of Cr⁺², Mn⁺², Co⁺², and Zn⁺²; and wherein M2 and M3 are each independently selected from the group consisting of Ni⁺², Ni⁺³, Mn⁺², Mn⁺³, Co⁺², Co⁺³, Cr⁺², and Zn⁺².
6. The superparamagnetic ferrite nanoparticles of any one of the above features, wherein the spinel crystal fields stabilize high spin states of the 3d-transition element cations in the d⁴, d⁵, or d⁶ orbitals, and the 3d-transition element cations are about 20%, about 40%, about 60%, or about 80% in high spin states.
7. The superparamagnetic ferrite nanoparticles of any one of the above features, wherein the magnetization of the superparamagnetic ferrite nanoparticles at 25° C. is about 115 emu/g.
8. The superparamagnetic ferrite nanoparticles of any one of the above features, wherein the the r₂ value at 25° C. is about 347 mM⁻¹S⁻¹ and the nanoparticles have a diameter of about 9 nm.
9. The superparamagnetic ferrite nanoparticles of any one of the above features, wherein SAR of the superparamagnetic ferrite nanoparticles in an alternating current (AC) magnetic excitation field of about 3.9 KA/m at a frequency of about 460 KHz is at least 514 Wg⁻¹.
10. The superparamagnetic ferrite nanoparticles of any of the above features, wherein about 20%, about 40%, about 60%, or about 80% of the 3d-transition metal cations have 4 or 5 unpaired d-orbital electrons.
11. The superparamagnetic ferrite nanoparticles of any one of the above features, wherein the nanoparticles have a spherical morphology.
12. The superparamagnetic ferrite nanoparticles of any one of the above features, wherein the nanoparticles have an average size from about 3 nm to about 50 nm.
13. The superparamagnetic ferrite nanoparticles of any one of the above features, wherein the nanoparticles have an average size from about 6 nm to about 15 nm.
14. The superparamagnetic ferrite nanoparticles of any one of the above features, wherein the nanoparticles have a particle size distribution with a geometric standard size deviation (σ) from about 0 to about 0.1.
15. The superparamagnetic ferrite nanoparticles of any one of the above features, wherein the nanoparticles comprise one or more coatings.
16. The superparamagnetic ferrite nanoparticles of feature 15, wherein the one or more coatings comprise one or more of metals, polymers, peptides, nucleotides, saccharides, ligands, lipids, and antibodies.
17. The superparamagnetic ferrite nanoparticles of feature 15 or feature 16, further comprising a targeting moiety.
18. The superparamagnetic ferrite nanoparticles of feature 17, wherein the targeting moiety binds to a tumor cell.
19. An imaging contrast agent comprising the superparamagnetic ferrite nanoparticles of any one of the above features.
20. The imaging contrast agent of feature 19, wherein the imaging contrast agent is an MRI contrast agent.
21. A magnetohypothermia agent comprising the superparamagnetic ferrite nanoparticles of any one of features 1-18.
22. The magnetohypothermia agent of feature 21, wherein the superparamagnetic ferrite nanoparticles have an average particle size of about 6 to 9 nm.
23. A diagnostic or therapeutic formulation for administration to a human or other mammalian subject, the formulation comprising one or more chelating agents and the superparamagnetic ferrite nanoparticles of any one of features 1-18.
24. A method of performing magnetohyperthermia on a subject, the method comprising the steps of:
(a) administering the superparamagnetic ferrite nanoparticles of any one of features 1-18 to the subject; and
(b) exposing the subject to a magnetic field, whereby a cell or tissue of the subject is heated.
   25. The method of feature 24, wherein the magnetic field is an alternating current (AC) magnetic excitation field having a frequency of about 100 to 500 KHz and an average field strength of Hf less than about 4.85×10⁹ Am⁻¹S⁻¹.
   26. The method of feature 24 or feature 25, wherein the cell or tissue of the subject is heated about 38° C. to about 45° C.
   27. The method of any one of features 24-26, wherein the superparamagnetic ferrite nanoparticles are targeted to tumor cells, and whereby at least a portion of the tumor cells is killed.
   28. A method of acquiring an MRI image utilizing a contrast agent, the method comprising the steps of:
(a) administering the superparamagnetic ferrite nanoparticles of any one of features 1-18 to a subject; and
(b) acquiring an MRI image of the subject; whereby the superparamagnetic ferrite nanoparticles enhance contrast in the MRI image.
   29. The method of feature 28, further comprising:
(c) performing magnetohyperthermia using the same superparamagnetic ferrite nanoparticles that were used for acquiring the MRI image.
   30. A method of making superparamagnetic ferrite nanoparticles of any one of features 1-18, the method comprising:
(a) providing reactants in the form of ions comprising 3d-transition elements M1, M2, and Fe and oxygen in a molar ratio (M1+M2)ₓFe_{y}O₄ wherein x=0 to 1, y=0 to 2.5, and x+y=3;
(b) heating the 3d-transition element cations, Fe, and oxygen in a reaction; and
(c) quenching the reaction, whereby said superparamagnetic ferrite nanoparticles are formed.
   31. The method of feature 30, wherein the heating is performed using a solution of said reactants and quenching is performed by raising pH of the solution or lowering the temperature of the solution.
   32. The method of feature 30 or feature 31, wherein the heating and/or quenching are performed under non-equilibrium conditions.
   33. The method of feature 32, wherein the non-equilibrium conditions comprise laser deposition, sputter deposition, ball milling, mechanochemical processing, heating followed by cooling, or addition of cation additives.
   34. The method of any one of features 30-33, further comprising rinsing, filtering, and drying the superparamagnetic ferrite nanoparticles.
   35. The method of feature 30, wherein:
the solution comprises 0.1 M iron (III) chloride hexahydrate and 0.05 M manganese (II) chloride;
heating comprises heating the solution to about 98° C.; and
quenching comprises adjusting pH of the solution by increasing [OH⁻] from 0.425 to 4.0 M over a period of about 120 minutes.

### BRIEF DESCRIPTION OF THE DRAWINGS

FIG. 1 shows a table listing the status of commercial MRI contrast agents based on European Medical Agency (EMA) suspensions of the marketing and sale of Gd-based CAs at 2017 [www.ema.europa.eu/medicines/human/referrals/gadolinium-containing-contrast-agents].

FIG. 1 shows a schematic illustration of an exemplary process of functionalization of superparamagnetic nanoparticles (SPM NPs) for liver tumor targeting utilizing arginylglycylaspartic acid (RGD) [2].

FIG. 2 shows low spin and high spin orbital filling for cations that can be present in superparamagnetic ferrite nanoparticles (SMFNs).

FIG. 3 shows a table of atomic, electronic, and magnetic properties of octahedrally coordinated Mn^{(2+/3+)} and Fe⁽³⁺⁾ cations in Mn-ferrite nanoparticles. Bohr magneton units (μ_{B}) are shown, and d-orbital (valence) filling is illustrated.

FIG. 4 shows a table of atomic, electronic, and magnetic properties of octahedrally coordinated Mn^{(2+/3+)} and Fe^{(2+/3+)} cations in Mn-ferrite nanoparticles. Bohr magneton units (μ_{B}) are shown correlated with d-orbital (valence) filling.

FIG. 5 shows the relaxivity (r2) of Feridex®, conventionally synthesized SMFNs (9 nm), and Quantum Spin Engineered (QSE) SMFNs (9 nm); the QSE SMFNs have a 31% enhancement compared to conventionally synthesized SMFNs.

FIG. 6 shows saturation magnetization as a function of estimated quench rate used during processing.

FIG. 7 shows a simulated plot of specific loss power (SLP) in watts/gram based on K and M for 12 nm nanoparticles. SLP of maghemite is indicated by the line with arrows on both ends. Simulations are based on the superparamagnetic characteristics of nanoparticles [4].

FIG. 8 shows the unit cell for the spinel ferrite structure showing 2 octahedra and 1 tetrahedron.

FIG. 9 shows a schematic of the bonding arrangement for superexchange interactions with a table (bottom) presenting GKA (Goodenough, Kanamori, Anderson) angles and exchange energies for the A-O—B and B—O—B bonding arrangements.

FIG. 10 shows the proposed paths to realizing Quantum Spin Engineered superparamagnetic nanoparticles (SPM NPs) for the co-optimization of MRI and magnetohypothermia (MHT).

FIG. 11 shows a plot of T_{N} (K), inversion parameter (fraction of high spin Mn residing on the octahedral sublattice), and molar concentration of NaOH (indicator of pH). High spin and low spin fields are indicated [15].

FIG. 12 shows a plot of T_{N} (K) vs. particle size. The inset in FIG. 12, a plot of TN(K) vs. Lattice Parameter (Å), illustrates impact upon lattice parameters as high spin (HS) and low spin (LS) radii (see FIGS. 3-4) are markedly different [15].

FIG. 13 shows a representative x-ray diffraction (XRD) θ-2θ spectrum for a Mn ferrite nanoparticle specimen. The data confirms that the nanoparticles are pure phase spinel ferrite. Least squares fitting analysis indicates the lattice parameter, a₀, is 0.84683±0.000183 nm. The inset is a TEM image of the same particles used in collection of these XRD data. The smaller features decorating the particles are residual surfactant used in processing [14].

FIG. 114A shows real part of the Fourier transformed Fe and Mn K edge EXAFS data with best fit data for a 7.5 nm diameter Mn-ferrite NP sample [15]. FIG. 14B shows Fe K edge XANES data with best linear combination fit for the same sample [14].

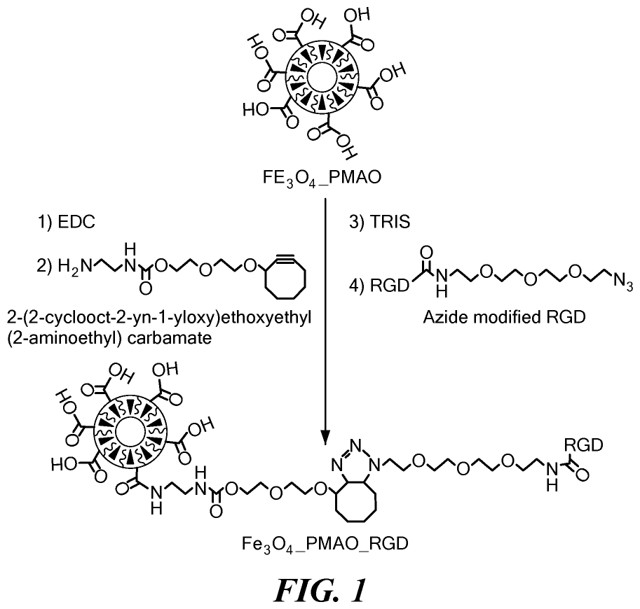

### DESCRIPTION

Concerns over gadolinium toxicity have led to urgent needs for engineering of new MRI contrast agents. After gadolinium, the 3d-transition element manganese (Mn) can possess the second highest paramagnetic moment of any element because the Mn⁺² cation has 5 unpaired electrons in its 3d shell. Detailed engineering the Mn⁺² cation can provide higher utility for MRI contrast agents while simultaneously providing magnetohyperthermia therapy (MHT) nanoparticles. The MHT nanoparticles can be used for sensitizing stubborn cancer cells (i.e. curing the incurable cancers). Methods of treating using the superparamagnetic ferrite nanoparticles herein with MHT can also enhance the efficacy of traditional therapeutic treatments, e.g., chemotherapy, and make possible the successful treatment of today's incurable cancers.

Stabilization of Mn⁺² ions, specifically in high spin states, while integrating the ions into a non-toxic form, which does not allow high concentrations of free Mn⁺² to circulate in a patient, can be accomplished by crystallizing the Mn⁺² in an engineered crystal form, for example, a spinel. Superparamagnetic ferrite nanoparticles (SMFNs) can be formed by engineering the spinel crystal forms in stoichiometric ranges described herein.

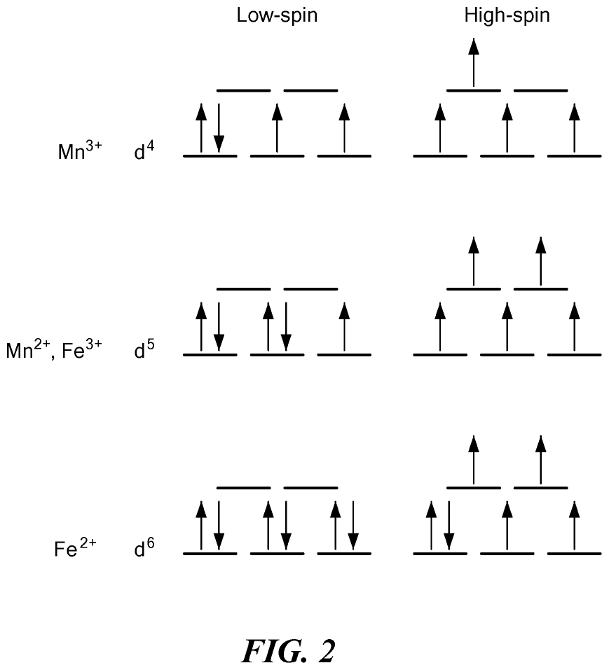

For example, stabilization of Mn⁺² in a high spin state (FIG. 2, center) can be accomplished by crystallizing Mn⁺² with Fe³⁺ in a quenched (non-equilibrium) crystallization reaction, forming spinel. The distance, direction, and angle of each of the cations with respect to the anion and the 2^{nd} cation can affect the strength of the exchange, and the spinel structures disclosed herein can enable stabilization of the high spin Mn⁺² with Fe³⁺ states, specifically when the cations are quenched into engineered positions in the crystal lattice during synthesis. Herein, the crystal lattice is being engineered to comprise distance, direction, and angle between any two cations to provide high MRI contrast and magnetohyperthermia capability. By engineering the syntheses to tailor the high spin states, a technique of Quantum Spin Engineering (QSE) is described herein to enable tailoring the MRI contrast agents and magnetohyperthermia therapy (MHT) nanoparticles. The MHT nanoparticles can be designed to produce heat when an alternating magnetic field is applied, enabling cancer treatments, while simultaneously providing high contrast MRI capability.

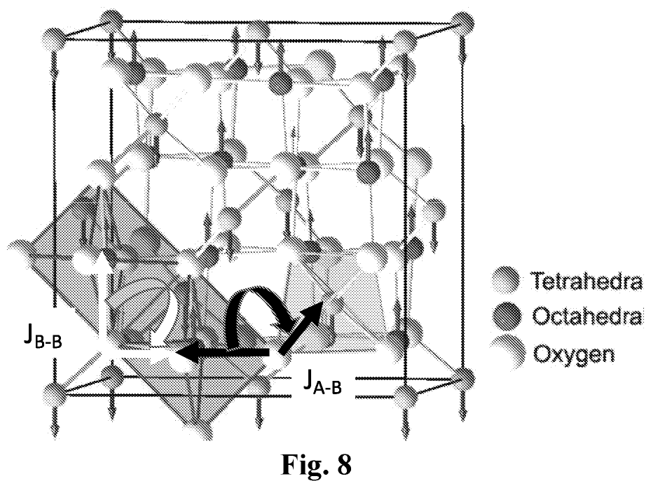

In the QSE designed spinel crystals, the direction and angle are defined by the relationship between Me-O-Me (Me=3d-transition cation), the critical distance is that between Me-O not Me-Me. All three factors (direction, angle, distance) can determine the degree of orbital overlap between the extending 2p orbitals of oxygen and the 3d orbitals of the cations (FIG. 8). The degree of orbital overlap can magnetically couple two 3d-transition element cations (with oxygen anion between) and can stabilize the high spin states of 3d-transition elements in the QSE (superparamagnetic) nanostructures described herein.

Superparamagnetic ferrite nanoparticles (SMFNs) are described herein along with methods of making SMFNs, methods of acquiring MRI images utilizing SMFNs as contrast agents, methods of treating patients utilizing SMFNs, and methods of targeting SMFNs to tumors or other areas. The QSE of Mn-ferrite herein was as proof of concept and with QSE of MnZn-ferrite and with expanded QSE into the reaches of spin engineering (e.g., utilizing 3d-transition elements), much higher r₂ values can be achieved compared to conventional MnZn-ferrite based CAs.

Making superparamagnetic ferrite nanoparticles (SMFNs) can begin with a mixture of 3d-transition elements and oxygen in a range of stoichiometry suitable for a spinel or other suitable crystal form.

Non-equilibrium crystallization can comprise introduction of increasing amounts of sodium hydroxide into an aqueous or nonaqueous precipitation reaction solution, by applying an increasing pH gradient to force (fast) precipitation and (fast) crystallization, resulting in a quenching of cations into non-equilibrium positions in a spinel crystal structure. Non-equilibrium positions means positions that cations would not normally occupy if the same crystal were to be grown under slow, gradial, equilibrium conditions. Non-equilibrium crystallization can comprise rapid cooling of a stoichiometric solution of cations and oxygen, for example, using a glass condenser to rapidly cool or quench. Rapid cooling of a solid phase stoichiometric spinal can induce Mn⁺² cations, for example, into non-equilibrium positions in a spinel crystal structure, for example, in high-speed ball milling (solid state). The non-equilibrium crystallization herein is a quenching comprising a quenching time, and as the quenching time is shortened, depending on reaction/crystallization conditions, particle size can decrease. While the examples provided herein can explain the Quantum Spin Engineering (QSM), any technique known in the art can be utilized to position high spin 3d-transition cations into high spin stabilized lattice positions in a spinel crystal structure.

Extended X-ray absorption fine structure (EXAFS) spectroscopy can be utilized to determine or confirm the positions of various cations in spinel or other crystal structures of superparamagnetic ferrite nanoparticles (SMFNs).

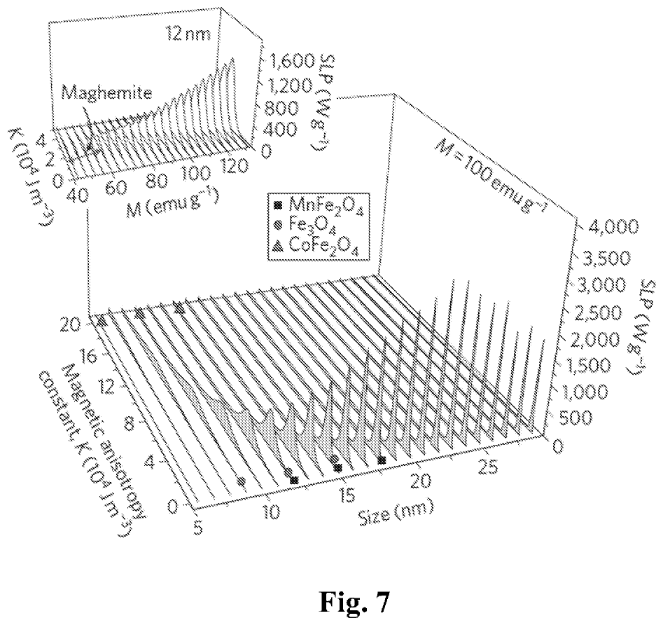

Particle size and uniformity of particle size of the superparamagnetic ferrite nanoparticles (SMFNs) can also be engineered during formation of SMFNs. In a modified NaOH co-precipitation method, the pH value of the solution can be adjusted by varying the molar amounts of [OH⁻]. As the pH is increased more rapidly, the quench time is reduced, and smaller particle sizes result. Additionally, the same conditions that lead to high magnetization lead to a reduction in particle size. With reduced particle size, the ratio of surface atoms to volumetric atoms increases allowing for the increased role of surface spin-canting and the surface “dead” layer thus reducing Ms. This enhancement continues as the quench rate increases until the spin canting dominates and reduces Ms (FIG. 7). As quench rate increases, quench time decreases. Quench times can be very short, for example, in a condensation crystallization from gas phase to solid phase. Depending on the non-equilibrium (quenched) conditions used to form SMFNs, quench time can be nanoseconds or milliseconds to hours; for example, up to 24 hours, up to 18 hours, up to 12 hours, up to 6 hours, up to 3 hours, up to 2 hours, or up to 1 hour.

When properly engineered, the SMFNs can show comparable performance to Gd-based contrast agents (GBCAs). The SMFNs can be in a stable and safe form (i.e., non-toxic and rapid renal clearance). SMFNs, being ferrites, do not dissolve in-vivo and hence do not suffer from associated side effects. Within SMFNs, Mn ions exist within a stable ferrite spinel crystal and are not susceptible to dissolution within the body's blood, tissue, or organs and therefore do not experience associated negative side effects. Additionally, SMFNs can be produced at roughly ⅕^{th} the cost of GBCAs, and being free of rare earth metals, experience an environmentally sustainable and secure global supply chain. Phase 2 activities of SMFNs include small animal studies of targeted imaging of implanted tumors, dual-use magnetic hyperthermia remediation of cancers, optimization of renal clearance rates, and cytotoxicity studies of conjugated SMFN agents.

The QSE nanoparticles herein, developed by the re-distribution of Mn²⁺ cations and 3d-transition cations from low spin to high spin sites produce a significant increase in the magnetization and relaxivity of nanoparticles, which can be employed as MRI contrast agents and simultaneously as MHT agents. This increase can directly improve the image contrast and resolution and therefore enhance the tumor detection sensitivity using this technology, while enabling treatments for tumors that could be previously untreatable. Further, the superparamagnetic ferrite nanoparticles described herein can apply to areas of a patient that were previously untreatable utilizing Gd-based CAs. QSE can produce CAs with higher efficiency, enabling the same superparamagnetic ferrite nanoparticles collected at the tumor sites for the detection as MRI contrast agent to simultaneously serve as the magnetic hyperthermia agents, i.e. triggering necrosis the cancerous cells and anti-angiogenesis in the tumor. Thus, the superparamagnetic ferrite nanoparticles herein can compete and possibly exceed the Gd-based commercial agents in the detection and treatment of tumor cells.

The superparamagnetic ferrite nanoparticles can be engineered by the controlled migration of Fe⁺² cations from low spin to high spin site, resulting in a major enhancement in the efficiency of nanoparticles used as magnetic hyperthermia agents. Available superparamagnetic iron oxide nanoparticles (SPIONs) have very poor MHT efficiency at the small size required for sufficient cellar uptake. This is due to the fact that the optimum Neel relaxivity of ferrite superparamagnetic nanoparticles at room temperature appear at large sizes of around 50 nm in diameter. This size is above the upper limit for superparamagnetic size of ferrite (<˜30 nm), therefore typical ferrite nanoparticles cannot experience their maximum performance in the superparamagnetic state. QSE maximizes the performance of the CAs, not only by enhancing the magnetization but also by bringing the room temperature optimum size (where the particles reaches its optimum performance) to the smaller size within the superparamagnetic region (about 7 nm to 15 nm), allowing for significantly higher MHT efficiency (and uptake) using such agents.

Shifting the optimum size to the smaller size region allows for achieving the optimum MRI resolution and MHT efficiency using smaller contrast agents, facilitating a high cellular uptake in the tumor cells and lead to a high tumor detection sensitivity compared with Gd-based CAs. The low cellular uptake, for example, due to particle size, of existing SPIONS is one of the main barriers for the application of SPIONs for cancer detection.

The present QSE technology offers a high (revolutionizing) tumor detection sensitivity via high magnetization and relaxivity leading to high image contrast and resolution, by shifting the maximum peak resolution to smaller size range, leading to higher tumor cellular uptake, and the QSE superparamagnetic NPs can be functionalized by targeting ligands, offering high selectivity by tumor cells and intracellular uptake.

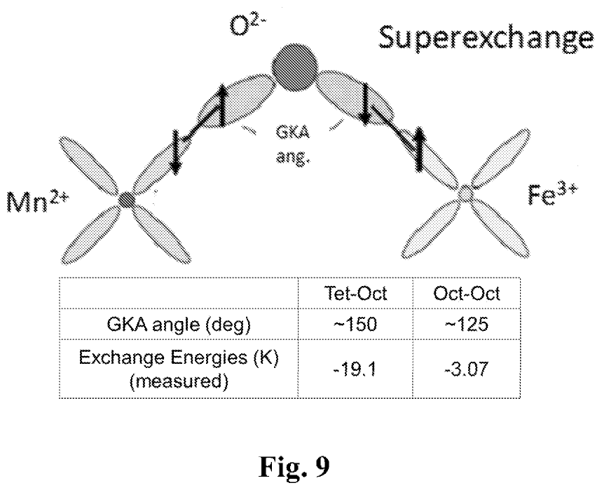

The magnetic properties of spinel ferrites (MeFe₂O₄, Me=Mn, Fe, Zn, or other 3d-transition element) can be determined by the type of divalent cation, Me²⁺, and its distribution on tetrahedral and octahedral sites in the spinel crystal structure. For example, partial substitution of zinc II cation (Zn⁺²) onto the thermodynamically preferred spinel tetrahedral coordination in Fe₃O₄ produces an increase in magnetic saturation at room temperature up to about 45% substitution after which the magnetic exchange weakens and the magnetic properties degrade. In this non-limiting example, Fe cation is placed into the octahedral positions as Zn occupies tetrahedral, with the angle of Me-O-Me (FIG. 9) planned for spin orbit coupling and high spin states. Nanosized crystallites can adopt different cation distributions compared to their bulk equivalents. The smaller as-synthesized nanocrystallites can have metastable cation inversion exhibiting higher saturation magnetization compared to their annealed and/or larger counterparts. This can lead to higher magnetization (Ms) and higher relaxation rates (r2). The higher r2 leads to enhanced MRI contrast leading to the ability to image early stage tumors. Spin-spin relaxation (r2) is approximately proportional to the square of Ms.

Quantum spin engineering (QSE) is the control of the cation distribution within spinel ferrite nanoparticle crystal structures from low to high spin states to tailor magnetic properties and to provide them with ultrasensitive imaging resolution and concomitant magnetic hyperthermia cancer remediation. QSE significantly impacts intrinsic magnetization and MRI relaxivity and allows for the tailoring of magnetic anisotropy for high sensitivity imaging and high efficiency magnetic hyperthermia (MHT) agents. While the examples herein discuss QSE applied to 3d-transition elements known in the art, the QSE technology can be utilized with any cation and any crystal structure to provide spin orbit coupling through oxygen anions or other suitable anions, stabilizing high spin states and providing high contrast MRI agents and MHT agents.

During typical crystal growth via precipitation, atoms or solute ions lodge into thermodynamically favorable open positions on the growing crystal during equilibrium growth where solute molecules or atoms precipitate out of solution. Typical crystal growth occurs at a rate wherein kinetics are such that cations are deposited in lower energy states in the crystal lattice. Crystal formation can be achieved by various methods, such as: seed crystals, change in pH, adding an organic or adding an antisolvent, solvent layering, sublimation, substitution of a cation or anion, cooling, or evaporation. During typical crystal growth, for example, in a precipitation reaction forming crystals, various cations will grow in equilibrium at thermodynamically favored positions, yet growth is governed by both thermodynamic and kinetic factors. Quenching discussed herein comprises non-equilibrium crystallization which can comprise any technique known in the art. Quench times discussed herein can comprise very short to long times because the crystallization conditions can vary widely, so long as the concepts of QSE provided herein are followed to stabilize high spin elemental states in a crystalline structure, to provide non-toxic (MRI and MHT) treatments for patients.

Cation distributions within the spinel superparamagnetic ferrite nanoparticles (SMFNs) herein can be tailored by adjusting the quench rate during chemical precipitation reactions, during crystallization, or during formation of spinel SMFNs. By adjusting the quench rate, non-equilibrium cation distributions can be engineered into crystalline ferrite nanoparticles (NPs) so that specific type 3d-transition (Me) cations and valency (e.g., 2+, 3+, etc.) reside on four-fold coordinated (tetrahedral or A) or six-fold coordinated (octahedral or B) spinel lattice sites with the goal of enhancing MRI or MHT properties or both simultaneously.

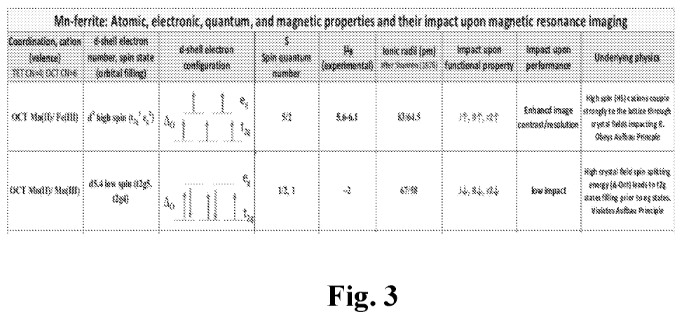

Addition of Zn⁺² as a cation additive can be used for increasing the Mn⁺² in octahedral positions and for reduction of the Fe⁺²/Fe⁺³ octahedral cation ratio (FIG. 9, right). Non-equilibrium cation distributions can be accomplished by adjusting the quench rate, for example, by employing a condenser during the reaction to chill (or to heat) the reactants, or by adjusting the pH during the reaction by adding buffering solutions. In this example, heat can be utilized to control the quench rate, for example, in cycles of a thermostat utilized to carefully control quench rate. A glassware condenser can be utilized during synthesis to adjust temperatures of reactants and to obtain SMFNs of different sizes that are processed as “fast quenched” and “slow quenched” reaction rates. As such, a quenching time can be defined for a planned set of reaction conditions. The quenching in of non-equilibrium cation distributions is dominated by “cation inversion” up to a quench rate for given conditions, then “spin canting” dominates as the quench rate is increased further (FIG. 7), in conditions where particle size decreases for faster quench. After crystallization, crystal fields (CF) acting on the A and B sites can stabilize high-spin (HS) versus low-spin (LS) configurations. Low spin and high spin orbital filling for cations present in superparamagnetic ferrite nanoparticles (SMFNs) are illustrated in FIG. 3.

By increasing the growth rate (decreasing the quenching time), the particles experience higher inversion parameters leading to a lower T_{N} (Neel temperature). The effect of increasing particle growth rate is akin to “quenching in” cation disorder, which has been reported in pulsed laser deposited ferrite films (e.g., from the Harris Team). Quenching utilizing nonequilibrium processing such as those that involve vapor quenching (e.g. pulsed laser deposition, sputter deposition) and high kinetic energy transfer (e.g. ball milling or mechanochemical processing), can provide high spin cations in spinel crystal structures. In chemical co-precipitation of a Mn-ferrite nanoparticle system or during synthesis of superparamagnetic ferrite nanoparticles (SMFNs) comprising 3d-transition elements, where ferrite nucleation and growth are believed to be of a low energy, pH determined growth rate can result in strong trends in cation disorder.

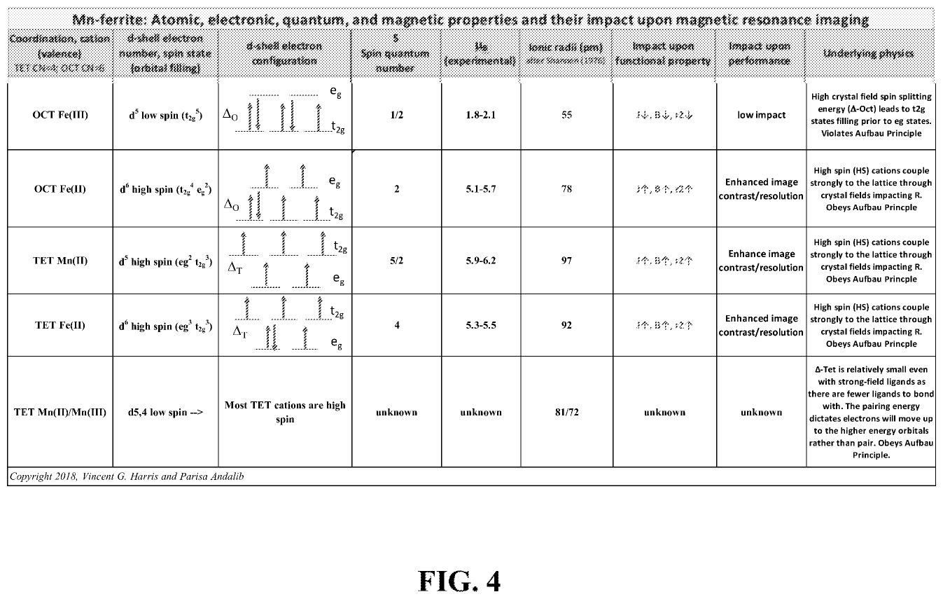

Selectivity of cation distributions to HS instead of to LS sites allows for the Quantum Spin Engineering (QSE) of ferrite NP contrast agents to possess intrinsic magnetization values that compete with the large moment of Gd-based contrast agents. For example, in review of the table shown in FIG. 3, the value for low spin (LS) Mn is ˜2 μ_{B} (Bohr magnetron units), high spin (HS) Mn approaches 6 μ_{B}, and Gd is ˜7 μ_{B}. In FIG. 4 the coordination number (CN) for a tetrahedral complex is 4, and the CN for an octahedral complex is 6. The table shown in FIG. 4 presents atomic, electronic, and magnetic properties of divalent and trivalent Mn and Fe cations coordinated in the octahedral lattice sites with high spin and low spin Hund's filling. In the HS configuration the CF energy D_{O} dominates the Hund's pairing energy sustaining the Aufbau's principle. The HS spin quantum number for both Mn²⁺ and Fe³⁺ is 5/2. The unpaired d-electrons give rise to the electron configuration per ion. In the LS configuration the Hund's pairing energy dominates the crystal fields (CF), and Aufbau's principle is maintained, and the number of unpaired spins in a minimum. The LS spin quantum number for both Mn²⁺ and Mn³⁺ is ½, and 1, respectively.

The table shown in FIG. 4 presents further atomic, electronic, and magnetic properties of divalent and trivalent Mn and Fe cations coordinated in the tetrahedral and octahedral lattice sites with high spin and low spin Hund's filling. The tables shown in FIGS. 3 and 4 represent novel concepts that can be utilized in QSE of other 3d-transition elements (e.g., cobalt, chromium). While the toxicity of cobalt can be an urgent concern for MRI and MHT treatments, the present technology provides concepts of QSE that can be applied for spin engineering of any suitable element into any suitable crystal form.

Because a tetrahedral complex has fewer ligands, the magnitude of the crystal field (CF) splitting is smaller. The difference between the energies of the t_{2g} and e_{g} orbitals in a tetrahedral complex (Dₜ) is slightly less ( 4/9) than half as large as the splitting in octahedral complexes (Dₒ). Mn⁺² is a high spin cation since it appears on the low end of the spectrochemical series of metals. Oxygen anion is a relatively weak ligand also supporting high spin Mn⁺² as either OCT or TET coordinated. Similar concepts hold for Fe⁺³.

However, Fe⁺² has large single ion anisotropy (high K) stemming from unquenched angular momentum. Because of this, tetrahedrally coordinated divalent iron leads to high K and high SAR (specific adsorption rate) resulting in more effective MHT agents. SAR is a parameter that can measure how effectively NPs generate heat to the tissue during magnetic hyperthermia treatment.

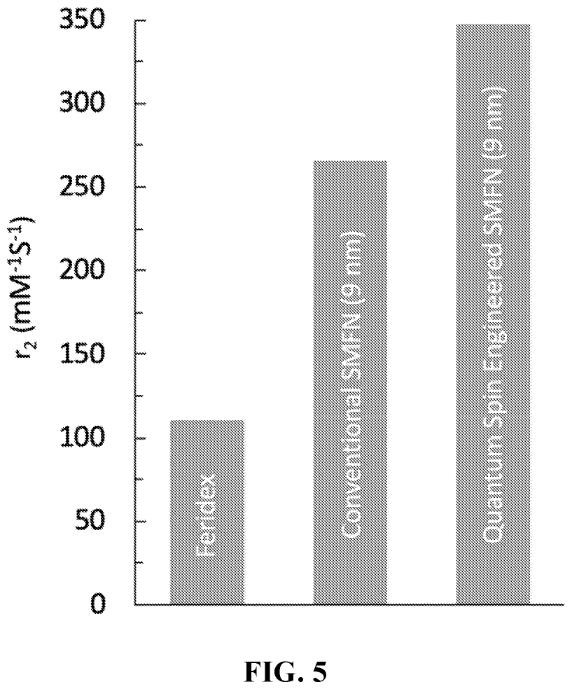

In superparamagnetic ferrite nanoparticles (SMFNs) samples produced by QSE, a 21% increase in room temperature magnetization (as Ms emu/g) over the typical values reported in the literature, (i.e. 95+/−5 emu/g) has been achieved, with the QSE SMFNs' value being 115.2 emu/g. SMFNs produced by QSE cannot be produced by a conventional, equilibrium synthesis. FIG. 5 shows the relaxivity (r2) of Feridexe, conventionally synthesized SMFNs (9 nm), and Quantum Spin Engineered (QSE) SMFNs (9 nm); the QSE SMFNs have a 31% enhancement compared to conventionally synthesized SMFNs.

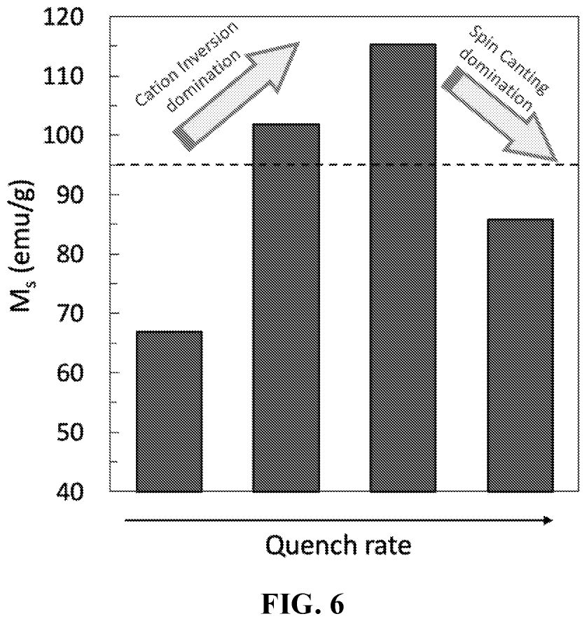

FIG. 6 provides a plot of the measured Ms values for each sample prepared, and saturation magnetization is shown as a function of estimated quench rate used during synthesis. FIG. 6 demonstrates that as quench rate increases the cations are “frozen” into nonequilibrium high spin sites, and cation inversion dominates. The modified spin configuration generates a higher magnetization and MRI response that is closely linked to the higher moment per cation due to unpaired spins and the imbalance of antialigned spinel sublattices. As the quench rate increases, cation disorder is “quenched in” the crystal structure and high spin states are stabilized in the crystal fields.

The same conditions that lead to high magnetization can lead to a reduction in particle size. With reduced particle size, the ratio of surface atoms to volumetric atoms increases allowing for the increased role of surface spin-canting and the surface “dead” layer thus reducing Ms. This enhancement continues as the quench rate increases until the spin canting dominates and reduces Ms. “Spin canting domination” is labeled in FIG. 6.

The relatively poor energy transfer efficiency from an excitation magnetic field to magnetohyperthermia therapy (MHT) nanoparticles (NPs) is a challenging obstacle for hyperthermia cancer treatment, which hinders efficacy and leads to the requirement for large doses of nanoparticles above toxicity thresholds. The heat generated by nanoparticles can depend on, for example, magnetic field, nanoparticle size, magnetization and magnetic anisotropy energy. A theoretical model proposed by Rosenweig shows that there is a sharp maximum in the specific loss power (SLP) indicating the optimal range of nanoparticle size and magnetic anisotropy. Since the magnetic anisotropy, K, is an intrinsic materials property, it is a challenging task to tune its values for optimal SLP. Here, K is optimized using, for the first time, cation spin engineering or QSE.

Based on the Rosenweig model [11], the SLP, which is the figure of merit for MHT of superparamagnetic nanoparticles, depends on spin relaxation processes, and therefore, obtains the dissipation relationships of dispersed superparamagnetic (SPM) particles based on rotational relaxation, resulting in the volumetric power dissipation P=μ₀ηfx″H₀² indicating that the dissipated power is only a function of SLP as follows,

$$SLP = \frac{P}{ρ ϕ} = \frac{{μ}_{0} π f {χ}^{″} {H}_{0}^{2}}{ρ ϕ} \qquad ( 1 )$$

where ρ is the particle density and ϕ is the nanoparticle volume fraction. Based on the relaxation equation of Shiliom is, the ferrofluid complex susceptibility is:

$$χ = {χ}^{′} + i {χ}^{″} = \frac{{χ}_{0}}{1 + {( ω τ )}^{2}} + i \frac{ω {τχ}_{0}}{1 + {( ω τ )}^{2}} \qquad ( 2 )$$

### Where

$${χ}_{0} = {χ}_{i} \frac{3}{ξ} ( coth ξ - \frac{1}{ξ} )$$

is the actual susceptibility,

$${χ}_{i} = \frac{{μ}_{0} ϕ {M}_{d}^{2} {V}_{m}}{3 kT}$$

is the susceptibility parameter and

$$ξ = \frac{{μ}_{0} {M}_{d} {H}_{0} {V}_{m}}{kT}$$

Langevin parameter, indicating that the power dissipation is maximum at

$$τ = \frac{1}{2 π f}$$

where the time constant effective relaxation time, originated from Brownian and Neel processes [11].

In FIG. 7, SLP is plotted as a function of magnetic anisotropy, K, diameter of the nanoparticle, D, and magnetization, M. There appears a sharp maximum in the curve, indicating the optimal range of K and D. In FIG. 7, the simulated plot of SLP based on K and M is for a 12 nm nanoparticle. In FIG. 7, the SLP of maghemite is indicated by the line with arrows on both ends. Simulations are based on the superparamagnetic characteristics of nanoparticles [4].

The inset at the upper left of FIG. 7 demonstrates the dependency of SLP on M. Enhancing the M can serve both purposes of enhancing SLP and T₂ for the purpose of MRI contrast enhancement (as discussed, T₂ is roughly proportional to the square of magnetization). This design parameter, and others, for example, K and D, can be enhanced using cation spin engineering for contrast enhancement and/or for MHT.

Utilizing Quantum Spin Engineering, a method of tuning the magnetic anisotropy (K) of the SPM nanoparticle to the desired value to obtain the highest heat rate is provided. Thus, by taking advantage of cation spin engineering proposed here, the magnetic properties of magnetic nanoparticles can be tuned to maximize the SLP.

In the spinel structure, metallic cations, of both magnetic and nonmagnetic nature, and typically divalent and trivalent ionic states, reside on the interstices of the close packed oxygen lattice, in some cases filling all available sites, while in others preferentially filling select sites.

The spinel structure can be expressed in the form [A]{B}₂O₄, where [ ] indicates divalent cations occupying four-fold tetrahedra lattice sites and { } indicates trivalent cations occupying six-fold octahedra lattice sites. [A]{B}₂O₄ is a single formula unit that constitutes ⅛ of a spinel unit cell, see FIG. 8. Cations have either four-fold or six-fold coordination forming tetrahedra (A) and octahedra (8) sublattices that are arranged in a close packed arrangement themselves. A cations reside on 8 of 64 available tetrahedral sites whereas B cations reside on 16 of 32 available octahedral sites. Oxygen anions form a close packed structure and contribute 32 ions that electrically balance the unit cell. FIG. 8 shows the unit cell for the spinel ferrite structure showing 2 octahedra and 1 tetrahedron.

Spinets are classified as normal, inverse, or mixed. The normal spinel has only divalent cations residing on the 8 of the available 64 A sites with trivalent ions filling the 16 of the 32 B sites. An example of a normal ferrite is the Zn spinel ferrite ([Zn^{II}]{Fe^{III}}₂O₄) where divalent Zn ions fill A-sites and trivalent Fe ions fill the B sites. Alternatively, when divalent ions fill 8 of the 16 B sites, with trivalent ions occupying the remaining A and B sites, an inverse spinel results. An example of an inverse spinel is Ni-ferrite ([Fe^{III}]{Ni_{0.5}^{II}Fe_{0.5}^{III}}₂O₄), another is Fe₃O₄ or [Fe^{III}]{Fe^{II}Fe^{III}}O₄.

Finally, the mixed spinel has different ions of mixed valence occupying both A and B sublattices. Because each species of ion has thermodynamically preferred lattice distributions based predominantly on cation ionic radius, electrostatic energy, and electronic configuration, this class of ferrite commonly results from nonequilibrium processing such as those that involve vapor quenching (e.g. pulsed laser deposition, sputter deposition) and high kinetic energy transfer (e.g. ball milling or mechanochemical processing), among other techniques. In chemical co-precipitation of a [[Mn-ferrite nanoparticle system]], where ferrite nucleation and growth are believed to be of a low energy, pH determined growth rate resulted in strong trends in cation disorder. Non-equilibrium cation distributions can be accomplished by adjusting the quench rate during crystal formation, for example, by employing a condenser during the reaction to chill or to heat the reactants, or by adjusting the pH during the reaction by adding buffering solutions. A glassware condenser can be utilized to quickly cool the reaction, quenching in non-equilibrium cation distributions. Faster quenching can lead to smaller particles, and spin canting dominates magnetism as the quench rate is increased further (FIG. 6).

Cation species that exist naturally in a multiplicity of valence states are more susceptible to this type of non-equilibrium structure. For example, Mn ions with common valences of 2, 3, 4, 5, 6, and 7, and are of comparable size to Fe ions, allow for such a distribution as [Mn^{2+,3+}_{1-δ}Fe^{2+,3+}_{δ}]{Mn^{2+,3+}_{δ}Fe^{2+,3+}_{2-δ}}O₄ to exist (where δ represents the cation inversion parameter). Thus, the present technology can be applied to any cation utilizing the QSE concepts provided herein.

The valence and distribution of the magnetic cation and its interaction with local crystal fields will determine its low spin or high spin nature. This in turn greatly impacts the magnetic exchange energy that affects their spin-spin and spin-lattice interaction and therefore relaxation and image contrast. The magnetic exchange energy can be planned and designed by considering cation and anion sizes, various crystal structures, orbitals and electron configurations of the cations, elements, and anions.

In considering the spinel ferrite systems, the magnetic exchange energy between spins of neighboring metallic ions is negative resulting in the antiparallel alignment of spins as the lowest energy configuration. Since the distance between metal ions is too great to support direct exchange, the exchange is mediated by the oxygen anion that resides between the two cations and thus superexchange is considered an indirect exchange (FIG. 9). Three factors principally affect the strength of the exchange, these include, the distance, direction, and angle of the cations with respect to the anion and the 2^{nd} cation. While the direction and angle are defined by the relationship between Me-O-Me, the critical distance is that between Me-O not Me-Me, the later having little impact on J. All three factors determine the degree of orbital overlap between the extending 2p orbitals of oxygen and the 3d orbitals of the cations and hence the magnitude of J.

Goodenough [Goodenough, 1955,1958] and Kanamori [Kanamori, 1959] put fourth phenomenological rules that explained the exchange strength as a function of bonding arrangements in magnetic oxide systems. Taken together, with the work of Anderson [Anderson, 1950], these rules now go by the name of Goodenough, Kanamori, Anderson (GKA) rules of superexchange. The GKA rules verify that larger Me-O-Me angles produce a larger orbital overlap integral leading to stronger exchange, while angles that approach 90° produce weak exchange (FIG. 9). This is seen in the ferrite systems where, for example, in spinel ferrites the exchange J_{AB} corresponds to the largest A-O—B angle, ˜150°, which is far larger than that of the B—O—B, J_{BB}, ˜125°. The A-O-A correlation forms an angle less than 80° and the distance between A-O is comparatively larger, ˜3.5 Å, leading to the smallest exchange, J_{AA}. J_{AA} is so comparatively small that many ignore it altogether. These relationships are depicted pictorially in FIG. 9, which is a schematic of the bonding arrangement for superexchange interactions. In the lower portion of FIG. 9 is a table presenting GKA angles and exchange energies for the A-O—B and B—O—B bonding arrangements. The larger Me-O-Me angles utilized herein can maximize the magnetic exchange energy between spins of neighboring metallic ions.

Combining the Me-O-Me angles and concepts of QSE discussed above with FIGS. 2-4 presents, for the first time, a collection of data highly relevant to the 3d-transition elements, Mn, and Fe cations in the Mn-ferrite system, or in the spinel superparamagnetic ferrite nanoparticle (SMFN) system. An examination of FIGS. 3-4 reveals the relationship between the ionic valence and electronic structure of the cations leading to high and low spin magnetic states. The concepts of QSE can then be applied to spin states as known in the art. A low magnetic spin state results from a crystal field energy that is too great to allow for Aufbau filling principles and therefore more electrons pair and limit the magnetic moment (that stems largely from the unpaired d-electrons). Alternatively, a high spin state has just the opposite physics, that is, the crystal fields are weak, and the electron filling follows Pauli exclusion and Aufbau principles. High spin cations are highly desirable for MRI and MHT purposes while low magnetic spin states are not. The far-right column of FIGS. 3-4 presents a brief explanation of the electronic filling at the core of these relationships. Utilizing the Me-O-Me angles of FIG. 9 stabilization, for example, of high spin cations of Mn²⁺ and Fe³⁺ on the octahedral sublattice of the spinel ferrite can obtain nearly 6 Bohr magnetons of magnetic moment and unprecedented T₂ contrast leading to exceptional MRI resolution. By combining these Quantum Spin Engineered nanoparticles with size engineering, for example, optimum MHT particles can be the same particles that provide unprecedented T₂ contrast leading to exceptional MRI resolution.

The collocation of Mn²⁺ and Fe³⁺ on octahedral sites (or other 3d-transition elements) leads to not only a distortion of the GKA bonding angles giving rise to increased exchange energy (J, signaled by changes in T_{N}) and magnetic flux density (B) and high T₂ contrast, but also tunes magnetic anisotropy energy (K) that has been shown (see FIG. 7) to provide very high SLP for effective magnetohyperthermia (MHT).

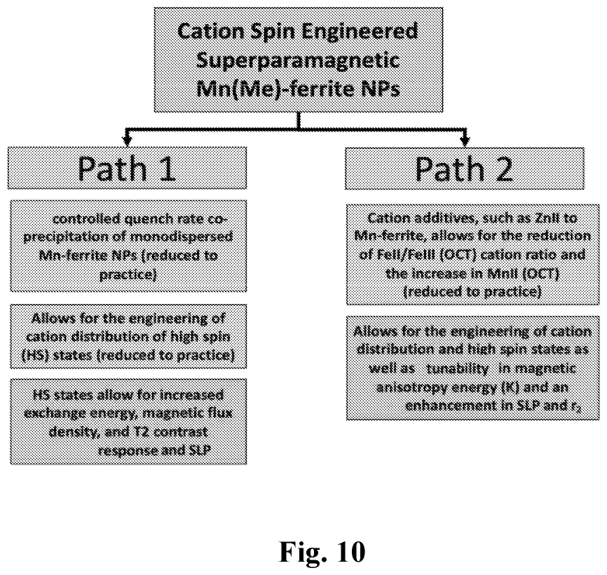

Non-limiting examples of paths to Quantum Spin Engineered SPM NPs for the co-optimization of MRI and MHT are shown in FIG. 10. One, see FIG. 10 (left), details the control of quench rate in the co-precipitation of the Mn-ferrite to tailor cation distribution. The other (FIG. 10, right) is the substitution of cations to displace Mn²⁺ from the tetrahedral sites to octahedral sites where they assume high spin electronic configurations.

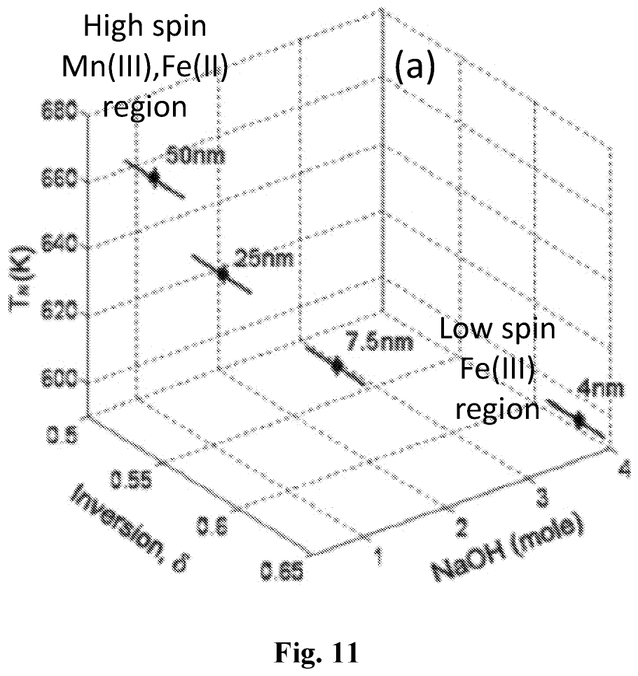

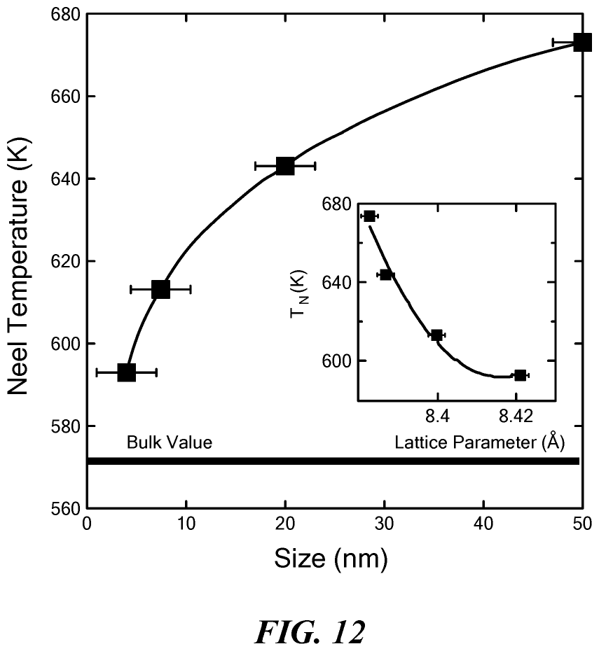

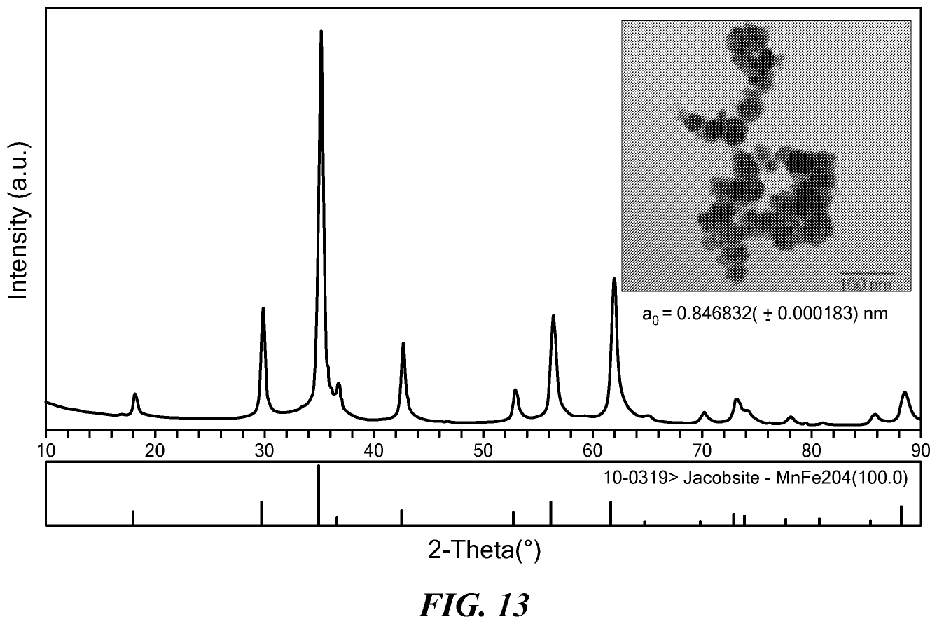

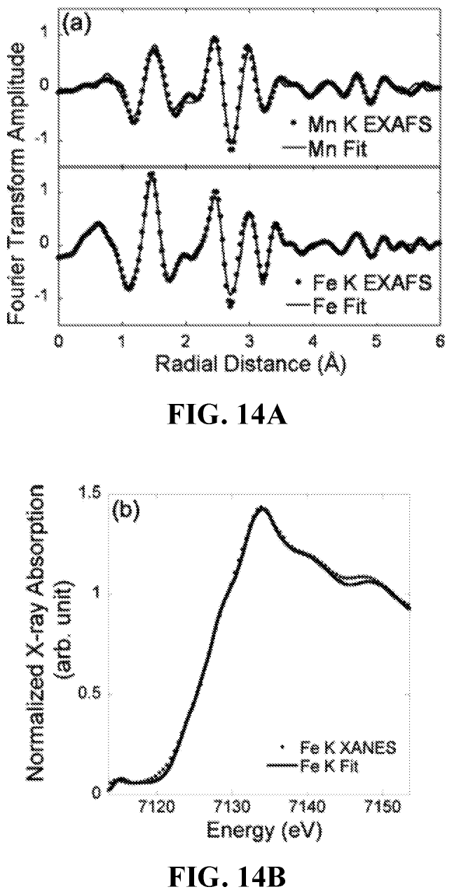

In “Path 1” of FIG. 10, the use of pH and temperature quench during co-precipitation of Mn-ferrite nanoparticles has been shown to alter both the cation distribution and Néel temperature. The Neel temperature reflects the magnetic exchange energy and naturally B (FIG. 11). Regions of high spin cation distribution and low spin cation distribution are indicated on FIG. 11. FIG. 11 shows a plot of T_{N} (K), inversion parameter (fraction of high spin Mn residing on the octahedral sublattice), and molar concentration of NaOH (indicator of pH). FIG. 12 shows a plot of T_{N} (K) vs. particle size. As indicated in FIG. 12, T_{N} increases with particle size. The inset in FIG. 12 illustrates impact upon lattice parameters as HS and LS radii (see FIGS. 3-4) are markedly different [15].

In “Path 2” of FIG. 10 (right) careful selection of substitutional cations is involved, and Path 2 is most about compositional design and less about processing. In this case, substituting Zn⁺² for the Mn⁺² during synthesis causes the Zn⁺² cations to reside solely on the tetrahedral spinel sublattice and to displace Mn⁺² cations to octahedral (high spin) sites. As a result, exchange energy increases (as does T_{N} and B) and T₂. The increased substitution of Zn continues to improve the system in this manner until the A-B exchange weakens from the population of nonmagnetic (Zn) cations and spin canting occurs and B decreases. The critical value of Zn that optimizes the MRI and MHT properties before degradation is about x=0.5.

The QSE processing of superparamagnetic monodispersed Mn ferrite nanoparticles is critical to achieving optimum MRI and MHT properties. Magnetic nanoparticles, like other types of inorganic materials, are developed with the expectation of product uniformity, reliable reproducibility, and property control based on manipulation of processing parameters. Due to the reduced dimensions of nanoparticles, however, each of these ordinary expectations is at risk of misinterpretation, variance, and/or inaccurate measurement. Ultimately, as technological advances applications require ever smaller device architectures, individual magnetic nanoparticles will be necessary for incorporation. For technological incorporation of magnetic nanoparticles into biomedical technologies such as MRI and MHT, chemical processing, control of composition, crystallinity, microstructure, morphology, phase, size, monodispersity, and functionalized coatings are necessary. The surface of QSE SMFNs modified using biocompatible polymer-based surfactants, e.g. polyethylene glycol (PEG) with tailored hydrodynamic and molecular properties for colloidal stability, uniform distribution, required reticuloendothelial system recognition, required circulation lifetime and clearance rate and optimum intracellular uptake of QSE-SMFNs. The scope of QSE as described herein is not limited by the examples provided and can be applied to, for example, planning coupling of d orbitals via intelligently designed crystalline forms, planning of particle sizes and shapes, planning of quench rates in synthesis and production, planning scale up, planning of magnetic properties, planning of coatings or layers of magnetic materials, co-crystals, and targeting. An advantage of the examples provided herein is the superparamagnetic properties can provide little to no magnetization and agglomeration of the particles when no external magnetic field is applied, which can be particularly useful for medical applications, but the concepts provided herein can be utilized to tune the Néel temperature for other applications besides medical applications, for example, paramagnetism at engineered temperatures.

Knowledge of the most sensitive processing parameters to the formation of the preferred phase and conditions to reduce agglomeration is necessary for reliable reproducibility. Control of the processing conditions is controlled for manipulation of the structural characteristics of the particles allowing control of the intrinsic magnetic properties. In an ideal sense, the chemical synthesis is controllable for all of these conditions without compromise. In practice, however, the design requires compromises to achieve the best possible set of characteristics. This is a difficult task requiring both careful process control with subsequent well thought out and thorough experimentation to characterize the particles. Chemical synthesis techniques can show great promise for producing the high-quality nanoparticles needed for applications.

Ferrite nanoparticles can be produced with a wide range of chemical and physical processing methods including: aqueous, polyol, micellar, sol-gel, and mechanical attrition, for example. Core-shell structures also can be produced that include such systems as spinel@iron, Au@Fe, Au@spinel, etc.

Superparamagnetic monodispersed Mn ferrite nanoparticles can be produced in a modified co-precipitation method. MnFe₂O₄ SPM nanoparticles were formed by dissolving 0.1 M of iron (III) chloride hexahydrate (FeCl₃.6H₂O, 99%, Sigma Aldrich) with 0.05 M of manganese (II) chloride (MnCl₂.4H₂O, 99%, Sigma Aldrich) in distilled water at a ratio of [Fe³⁺]/[Mn²⁺]=2:1 to maintain the stoichiometry of the spinel ferrite. The mixed metal chloride precursor solution was introduced slowly into a boiling NaOH solution and the reaction was carried out at 98° C. for 120 min. The pH value of the solution was adjusted by varying the molar amounts of [OH⁻] from 0.425 to 4.0 M. The pH environment is critical to particle nucleation, rate of growth, and stabilization, and ultimately the size of the resulting particles. Following the completion of the reaction, the resulting particles were rinsed in distilled water, filtered, and dried at 70° C. for 12 hours.

The SMP NP samples have been studied by extended x-ray absorption fine structure (EXAFS) spectroscopy to show measurement of cation distribution in the spinel crystal. Importantly, the potential of EXAFS to measure element specific cation distributions allowing for the investigation of their impact upon intrinsic magnetic properties in ferrite systems is shown in FIGS. 18A-18B.

In all samples, the EXAFS modeling (see FIGS. 18A-18B) indicated the inversion parameter δ, i.e. (Mn_{1-δ}Fe_{δ})^{Tet}(Mn_{δ} Fe_{2-δ})^{Oct}O₄, to be higher than the 20%, typically quoted as the equilibrium value. As the particle size increases, δ decreased from 62.9+/−1.8% to 54.2+/−2.3%. From FIGS. 18A-18B, it is observed that the highest δ corresponds to both the lowest T_{N} and the highest molar concentration of NaOH (highest pH). The latter correlates directly with NP growth rate. FIG. 14A shows real part of the Fourier transformed Fe and Mn K edge EXAFS data with best fit data for 7.5 nm diameter Mn-ferrite NP sample. FIG. 14B shows Fe K edge XANES data with best linear combination fit for the same sample.

By increasing the growth rate, the particles can experience higher inversion parameters leading to a lower T_{N}. The effect of increasing particle growth rate is akin to “quenching in” cation disorder, which can also be in pulsed laser deposited ferrite films [Harris Team]. From the EXAFS fitting results, a higher mean square radial displacement relative to Mn ions was measured, consistent with there being multiple oxidation states within the structure. The particle surface chemistry, structure, magnetic spin configuration, and/or finite scaling, play secondary roles. A representative best fit to Fe K edge XANES are shown in FIG. 14B. The fitting of the Mn XANES indicates a distribution of 55% divalent, 35% trivalent, and 10% quadrivalent ions. The appearance of quadrivalent Mn can indicate the presence of cation defects.

Methods to achieve uniform size distribution of cation spin engineered (or QSE) nanoparticles are provided herein. Simultaneously, methods to determine the optimum size of cation spin engineered nanoparticles are provided, for example, while also considering optimum K, D, and M values.

Based on the foregoing discussion, high spin (i.e. Fe³⁺−Oct, Mn²⁺−Oct) cations can be stabilized within uniquely engineered superparamagnetic ferrite nanoparticles (SMFNs) and Mn(Me)-ferrite (e.g. Me=Zn) nanoparticle. The same cation distribution optimizes the magnetic anisotropy value (K). The technology simultaneously can engineer the ionic and electronic structures of superparamagnetic ferrite nanoparticles (SMFNs) and Mn-based ferrite SPMs to provide dual-purpose optimization where both imaging and cancer remediation are advanced beyond the previous state of technology.

The technologies herein provide a financial advantage and are about ⅕th the cost of Gd-based CAs (as confirmed by online cost estimates). The process has an environmentally sustainable and secure U.S. supply chain as opposed to rare earth elements, e.g., Gd, required for production of Gd-based CAs. Almost all rare-earth element supply chains originate in far reaches and are strictly regulated to the community's detriment. The QSE-NP processing herein is scalable to low-cost large-scale (i.e., industrial) production.

As used herein, the term “about” and “approximately” mean close to the stated value, as understood by one of ordinary skill in the art. For example, the terms “about” and “approximately” can mean within 10%, within 5%, within 1%, or within 0.5%.

As used herein, “consisting essentially of” allows the inclusion of materials or steps that do not materially affect the basic and novel characteristics of the claim. Any recitation herein of the term “comprising”, particularly in a description of components of a composition or in a description of elements of a device, can be exchanged with the alternative expressions “consisting essentially of” or “consisting of”.

### REFERENCES

1. P. W. Anderson (1950). “Antiferromagnetism. Theory of Superexchange Interaction”. Phys. Rev. 79 (2): 350. doi:10.1103/PhysRev.79.350.
2. Arriortua, O. K.; Garaio, E.; Herrero de la Parte, B.; Insausti, M.; Lezama, L.; Plazaola, F.; Garcia, J. A.; Aizpurua, J. M.; Sagartzazu, M.; Irazola, M.; Etxebarria, N.; Garcia-Alonso, I.; Saiz-López, A.; Echevarria-Uraga, J. J. Beilstein J. Nanotechnol. 2016, 7, 1532-1542. doi:10.3762/bjnano.7.147.
3. C. N. Chinnasamy, Aria Yang, S. D. Yoon, Kailin Hsu, M. D. Shultz, E. E. Carpenter, S. Mukerjee, C. Vittoria, and V. G. Harris, “Size dependent magnetic properties and cation inversion in chemically synthesized MnFe2O4 nanoparticles,” J. Appl. Phys. 101, 09M509 (2007).
4. J. Cheon et al., Exchange-coupled magnetic nanoparticles for efficient heat induction, Nature Nanotech. 6. (2011) 418-422.
5. J. B. Goodenough (1955). “Theory of the Role of Covalence in the Spinel-Type Manganites [La, M(II)]MnO3”. Phys. Rev. 100 (2): 564. doi:10.1103/PhysRev.100.564.
6. John B. Goodenough (1958). “An interpretation of the magnetic properties of the spinel-type mixed crystals La1-xSrxCoO3-λ”. J. Phys. Chem. Solids. 6 (2-3): 287. doi:10.1016/0022-3697(58)90107-0.
7. V. G. Harris, N. C. Koon, C. M. Williams, Q. Zhang, M. Abe, and J. Kirkland, “Cation distributions in spinel ferrites via EXAFS,” Appl. Phys. Lett., 68 (15), 2082 (1996).
8. V. G. Harris, N. C. Koon, C. M. Williams, Q. Zhang, M. Abe, J. P. Kirkland, and D. A. McKeown, “Direct measurement of octahedral and tetrahedral site environments in NiZn-ferrites,” IEEE Trans. on Magn., 31, No. 6, 3473 (1995).
9. J. Kanamori (1959). “Superexchange interaction and symmetry properties of electron orbitals”. J. Phys. Chem. Solids. 10 (2-3): 87. doi:10.1016/0022-3697(59)90061-7.
10. Joshua E. Rosen, Lorena Chan, Dar-Bin Shieh, Frank X. Gu, Iron oxide nanoparticles for targeted cancer imaging and diagnostics, Nanomedicine: Nanotechnology, Biology and Medicine, Volume 8, Issue 3, 2012, pages 275-290,doi:10.1016/j.nano.2011.08.017.
11. R. E. Rosensweig, heating magnetic fluid with alternating magnetic field, Journal of Magn. and Magn. Mat. 252 (2002) 370-374.
12. Splendiani, A., Perri, M., Marsecano, C. et al. Radiol med (2018) 123: 125. doi.org/10.1007/s11547-017-08169.
13. M. A. Willard, L. K. Kurihara, E. E. Carpenter, S. Calvin, and V. G. Harris, “Chemically prepared magnetic nanoparticles,” International Materials Reviews, vol. 49, 2004, pp. 125-170.
14. Aria Yang, Ch. Chinnasamy, G. M. Greneche, Kailin Hsu, C. Vittoria, and V. G. Harris, “Enhanced Neel temperature in Mn-ferrite nanoparticles linked to growth rate induced cation inversion,” Nanotechnology, 20, 185704 (2009).
15. Aria Yang, C. N. Chinnasamy, J. M. Greneche, Yajie Chen, Soack D. Yoon, Kailin Hsu, C. Vittoria and V. G. Harris, “Large tunability of Neel temperature by growth-rate-induced cation inversion in Mn-ferrite nanoparticles,” Appl. Phys. Lett., 94, 113109 (2009).
16. Yu, W. W.; Chang, E.; Sayes, C. M.; Drezek, R.; Colvin, V. L. Nanotechnology 2006, 17, 4483-4487. doi:10.1088/0957-4484/17/17/033.
17. Xu Zuo, Aria Yang, Soack-Dae Yoon, Joseph A. Christodoulides, Vincent G. Harris, and Carmine Vittoria, “Large induced magnetic anisotropy in manganese spinel ferrite films,” Appl. Phys. Lett. 87, 152505 (2005).

1. Superparamagnetic manganese ferrite nanoparticles comprising a spinel compound having a crystal structure represented by
[Mn_{1-δ}^{+2,+3}+Fe_{δ}^{+2,+3}]ₓ^{Tet}{Fe_{1-δ}^{+2,+3}+Mn_{δ}^{+2,+3}}_{y}^{Oct}O₄
wherein δ is a cation inversion parameter representing a population of Mn ions on octahedral sites; wherein x is 0.8 to 1.2, y is 1.8 to 2.2, and x+y=3; wherein Fe^{+2, +3} comprises Fe⁺² or Fe⁺³, and wherein Tet designates a spinel tetrahedral site and Oct designates a spinel octahedral site.

2. Superparamagnetic zinc ferrite nanoparticles comprising a spinel compound having a crystal structure represented by
[Zn_{1-ϕ}⁺²+Fe_{ϕ}^{+2,+3}]ₓ^{Tet}{Fe_{1-ϕ}^{+2,+3}+Zn_{ϕ}⁺²}_{y}^{Oct}O₄
wherein ϕ is a cation inversion parameter representing a population of Zn ions on octahedral sites; wherein x is 0.8 to 1.2, y is 1.8 to 2.2, and x+y=3; wherein Fe^{+2, +3} comprises Fe⁺² or Fe⁺³, and wherein Tet designates a spinel tetrahedral site and Oct designates a spinel octahedral site.

3. Superparamagnetic manganese zinc ferrite nanoparticles comprising a spinel compound having a crystal structure represented by
[Zn⁺²_{1-ϕ}+Mn_{1-δ-ϕ}^{+2,+3}+Fe_{δ-ϕ}^{+2,+3}]ₓ^{Tet}{Fe_{1-δ-ϕ}^{+2,+3}+Mn_{δ-ϕ}^{+2,+3}Zn⁺²_{ϕ}}_{y}^{Oct}O₄
wherein δ is a cation inversion parameter representing a population of Mn ions on octahedral sites; wherein ϕ is a cation inversion parameter representing a population of Zn ions on octahedral sites; wherein x is 0 to 1.25, y is 0.75 to 3, and x+y=3; wherein Fe^{+2, +3} comprises Fe⁺² or Fe⁺³, and wherein Tet designates a spinel tetrahedral site and Oct designates a spinel octahedral site.

4. Superparamagnetic mixed ferrite nanoparticles comprising a spinel compound having a crystal structure represented by
[AM1⁺²_{1-ϕ}+DM2_{1-δ-ϕ}⁺²+(1−D)M2_{1-δ-ϕ}⁺³+EFe_{δ-ϕ}⁺²+(1−E)Fe_{δ-ϕ}⁺³]ₓ^{Tet}{GFe_{1-δ-ϕ}⁺²+(1−G)Fe_{1-δ-ϕ}⁺³+JM2_{δ-ϕ}⁺²+(1−J)M2_{δ-ϕ}⁺³+(1−A)M1⁺²_{ϕ}}_{y}^{Oct}O₄
wherein M1 is a divalent cation, such as a cation of Zn, Cu, Mg, or Cr;
wherein M2 is a mixed valence 3d transition metal cation, such as a cation of a transition metal such as Mn, Fe, Ni, or Co;
wherein A=0 to 1, (D+J)=0 to 1, (E+G)=0 to 1, and x+y=3;
wherein δ is a cation inversion parameter representing a population of Mn ions on octahedral sites;
wherein ϕ is a cation inversion parameter representing a population of Zn ions on octahedral sites;
wherein Fe^{+2, +3} comprises Fe⁺² or Fe⁺³, and
wherein Tet designates a spinel tetrahedral site and Oct designates a spinel octahedral site.

5. The superparamagnetic mixed ferrite nanoparticles of claim 4,
wherein M1 is selected from the group consisting of Cr⁺², Mn⁺², Co⁺², and Zn⁺²; and
wherein M2 and M3 are each independently selected from the group consisting of Ni⁺², Ni⁺³, Mn⁺², Mn⁺³, Co⁺², Co⁺³, Cr⁺², and Zn⁺².

6. The superparamagnetic ferrite nanoparticles of any one of the above claims, wherein the spinel crystal fields stabilize high spin states of the 3d-transition element cations in the d⁴, d⁵, or d⁶ orbitals, and the 3d-transition element cations are about 20%, about 40%, about 60%, or about 80% in high spin states.

7. The superparamagnetic ferrite nanoparticles of any one of the above claims, wherein the magnetization of the superparamagnetic ferrite nanoparticles at 25° C. is about 115 emu/g.

8. The superparamagnetic ferrite nanoparticles of any one of the above claims, wherein the the r₂ value at 25° C. is about 347 mM⁻¹S⁻¹ and the nanoparticles have a diameter of about 9 nm.

9. The superparamagnetic ferrite nanoparticles of any one of the above claims, wherein SAR of the superparamagnetic ferrite nanoparticles in an alternating current (AC) magnetic excitation field of about 3.9 KA/m at a frequency of about 460 KHz is at least 514 Wg⁻¹.

10. The superparamagnetic ferrite nanoparticles of any of the above claims, wherein about 20%, about 40%, about 60%, or about 80% of the 3d-transition metal cations have 4 or 5 unpaired d-orbital electrons.

11. The superparamagnetic ferrite nanoparticles of any one of the above claims, wherein the nanoparticles have a spherical morphology.

12. The superparamagnetic ferrite nanoparticles of any one of the above claims, wherein the nanoparticles have an average size from about 3 nm to about 50 nm.

13. The superparamagnetic ferrite nanoparticles of any one of the above claims, wherein the nanoparticles have an average size from about 6 nm to about 15 nm.

14. The superparamagnetic ferrite nanoparticles of any one of the above claims, wherein the nanoparticles have a particle size distribution with a geometric standard size deviation (σ) from about 0 to about 0.1.

15. The superparamagnetic ferrite nanoparticles of any one of the above claims, wherein the nanoparticles comprise one or more coatings.

16. The superparamagnetic ferrite nanoparticles of claim 15, wherein the one or more coatings comprise one or more of metals, polymers, peptides, nucleotides, saccharides, ligands, lipids, and antibodies.

17. The superparamagnetic ferrite nanoparticles of claim 15 or claim 16, further comprising a targeting moiety.

18. The superparamagnetic ferrite nanoparticles of claim 17, wherein the targeting moiety binds to a tumor cell.

19. An imaging contrast agent comprising the superparamagnetic ferrite nanoparticles of any one of the above claims.

20. The imaging contrast agent of claim 19, wherein the imaging contrast agent is an MRI contrast agent.

21. A magnetohypothermia agent comprising the superparamagnetic ferrite nanoparticles of any one of claims 1-18.

22. The magnetohypothermia agent of claim 21, wherein the superparamagnetic ferrite nanoparticles have an average particle size of about 6 to 9 nm.

23. A diagnostic or therapeutic formulation for administration to a human or other mammalian subject, the formulation comprising one or more chelating agents and the superparamagnetic ferrite nanoparticles of any one of claims 1-18.

24. A method of performing magnetohyperthermia on a subject, the method comprising the steps of:
(a) administering the superparamagnetic ferrite nanoparticles of any one of claims 1-18 to the subject; and
(b) exposing the subject to a magnetic field, whereby a cell or tissue of the subject is heated.

25. The method of claim 24, wherein the magnetic field is an alternating current (AC) magnetic excitation field having a frequency of about 100 to 500 KHz and an average field strength of Hf less than about 4.85×10⁹ Am⁻¹S⁻¹.

26. The method of claim 24 or claim 25, wherein the cell or tissue of the subject is heated about 38° C. to about 45° C.

27. The method of any one of claims 24-26, wherein the superparamagnetic ferrite nanoparticles are targeted to tumor cells, and whereby at least a portion of the tumor cells is killed.

28. A method of acquiring an MRI image utilizing a contrast agent, the method comprising the steps of:
(a) administering the superparamagnetic ferrite nanoparticles of any one of claims 1-18 to a subject; and
(b) acquiring an MRI image of the subject; whereby the superparamagnetic ferrite nanoparticles enhance contrast in the MRI image.

29. The method of claim 28, further comprising:
(c) performing magnetohyperthermia using the same superparamagnetic ferrite nanoparticles that were used for acquiring the MRI image.

30. A method of making superparamagnetic ferrite nanoparticles of any one of claims 1-18, the method comprising:
(a) providing reactants in the form of ions comprising 3d-transition elements M1, M2, and Fe and oxygen in a molar ratio (M1+M2)ₓFe_{y}O₄ wherein x=0 to 1, y=0 to 2.5, and x+y=3;
(b) heating the 3d-transition element cations, Fe, and oxygen in a reaction; and
(c) quenching the reaction, whereby said superparamagnetic ferrite nanoparticles are formed.

31. The method of claim 30, wherein the heating is performed using a solution of said reactants and quenching is performed by raising pH of the solution or lowering the temperature of the solution.

32. The method of claim 30 or claim 31, wherein the heating and/or quenching are performed under non-equilibrium conditions.

33. The method of claim 32, wherein the non-equilibrium conditions comprise laser deposition, sputter deposition, ball milling, mechanochemical processing, heating followed by cooling, or addition of cation additives.

34. The method of any one of claims 30-33, further comprising rinsing, filtering, and drying the superparamagnetic ferrite nanoparticles.

35. The method of claim 30, wherein:
the solution comprises 0.1 M iron (III) chloride hexahydrate and 0.05 M manganese (II) chloride;
heating comprises heating the solution to about 98° C.; and
quenching comprises adjusting pH of the solution by increasing [OH⁻] from 0.425 to 4.0 M over a period of about 120 minutes.
